(19) United States
(12) Patent Application Publication
(10) Pub. No.: US 2021/0353950 A1
(43) Pub. Date: Nov. 18, 2021

(54) SAFETY ASSEMBLY FOR PERFORMING ENDODERMAL RADIOFREQUENC Y TREATMENTS

(71) Applicants: Roberta MAZZANTI, (US); TARAS S.R.L., Bologna (IT)

(72) Inventor: Pietro Tomaso GRAMOLI, Loiano (IT)

(21) Appl. No.: 17/287,658

(22) PCT Filed: Oct. 23, 2018
(86) PCT No.: PCT/IT2018/000135

§ 371 Date: Apr. 22, 2021

(51) Int. Cl. A61N 1/40

(52) U.S. Cl. CPC A61N 1/403

(57) ABSTRACT

A safety assembly for performing endodermal radiofrequency treatments, performed by a needle-shaped transducer supplied by a respective control and management unit and a cannula, made of electrically conducting material and partially superficially insulated, designed to accommodate the needle-shaped transducer and intended to be arranged under the skin of the patient. The assembly includes an identification element, uniquely associated with the cannula, which incorporates a first unique data string. The control and management unit includes a reader for reading the first data string of the identification element; the reader is designed to enable the operation of the transducer upon reading a correct first data string.

### TECHNICAL FIELD

The present disclosure relates to a safety assembly for performing endodermal radiofrequency treatments.

### BACKGROUND

Radiofrequency aesthetic/medical therapy utilizes the effect generated by adapted electromedical devices, which produces a “remodeling” of tissues, with an action that is particularly effective in contrasting skin relaxation of the face and body.

The principle underlying radiofrequency is the generation of a thermal shock in the deepest layers of the skin in order to trigger a regenerative response on the part of the body. The radiofrequency electromagnetic field is generated by a high-frequency (above 100 kHz) alternating electric current, the flow of which changes direction very rapidly, and which does not stimulate the nerve and muscle tissues but has a controlled “thermal effect” by temperature increase.

Radiofrequency treatments can also be performed in an endodermal mode.

By means of a specific needle cannula which is made of electrically conducting material and is partially insulated superficially (within which a transducer is accommodated which is connected to a main apparatus for control, management and power supply, capable of establishing the variable-frequency electrical field), it is possible to perform the radiofrequency treatment directly on the tissues of interest (under the skin of the patient).

This is possible by making the needle cannula pass through the dermis (or a predefined portion thereof) so that it can slide along a surface that is parallel to the skin of the patient but inside it.

This type of treatment is used in aesthetic medicine in order to perform liposculpture procedures on the patient, which allow to reduce (and even eliminate) imperfections, defects and other problems even of a functional nature.

This type of treatment requires each needle cannula to be sterile in order to avoid contaminations among patients.

The material by means of which the needle cannulas are provided is not suitable for sterilization in an autoclave and for this reason it is preferred to resort to components of the disposable type.

In practice, therefore, at the end of the treatment performed on a patient, the needle cannula must be eliminated (discarded in the trash) without the possibility to reuse it.

This method is entrusted to the skill of each operator and therefore can be ignored by someone who wishes to use the same needle cannula multiple times (subjecting however the patients to a potential danger situation).

The transducers, too, have an upper use time limit and for this reason must be replaced periodically. Failed replacement might cause the emission of a variable magnetic field which is unsuitable for the purpose and/or the impossibility to detect the temperature in a precise manner, with the consequent impossibility of a respective feedback adjustment thereof. The replacement of the transducer also depends solely on the skill of the operator.

Another potential source of risk for the patient lies in the possibility that the coupling between the needle cannula and the transducer is not stable or is not correct: any sliding of the transducer within the needle cannula entails an unreliable temperature detection, with possible risks of burns for the patient; the coupling of a transducer that is excessively short for a given needle cannula in turn can cause thermal effect problems or can cause ineffectiveness of the treatment.

### SUMMARY

The aim of the present disclosure is to solve the problems described above, by proposing a safety assembly for performing endodermal radiofrequency treatments that ensures the single use of each needle cannula, safeguarding the safety of the patients.

Within this aim, the disclosure proposes a safety assembly for performing endodermal radiofrequency treatments that ensures the correct replacement of each transducer when it has reached the respective planned life limit.

The disclosure also proposes a safety assembly for performing endodermal radiofrequency treatments that ensures optimum and correct coupling of each individual transducer with the corresponding needle cannula.

The present disclosure further provides a safety assembly for performing endodermal radiofrequency treatments that have low costs, is relatively simple to provide in practice and is safe in application.

This aim and these advantages, as well as others which will become is better apparent hereinafter, are achieved by providing a safety assembly for performing endodermal radiofrequency treatments, performed by means of a needle-shaped transducer supplied by a respective control and management unit and a cannula, made of an electrically conducting material and partially superficially insulated, designed to accommodate said needle-shaped transducer and intended to be arranged under the skin of the patient, characterized in that it comprises an identification element, uniquely associated with said cannula, which incorporates a first unique data string, said control and management unit comprising a reader for reading at least said first data string of said identification element, said reader being designed to enable the operation of said transducer upon reading a correct first data string.

### BRIEF DESCRIPTION OF THE DRAWINGS

Further characteristics and advantages of the disclosure will become better apparent from the description of a preferred but not exclusive embodiment of the safety assembly for performing endodermal radiofrequency treatments according to the disclosure, illustrated by way of nonlimiting example in the accompanying drawings, wherein:

FIG. 1 is an exploded schematic perspective view of some components of a safety assembly for performing endodermal radiofrequency treatments according to the disclosure;

FIG. 2 is a schematic perspective view of the components of FIG. 1 in a partially coupled configuration; and

FIG. 3 is a schematic perspective view of the assembly according to the disclosure.

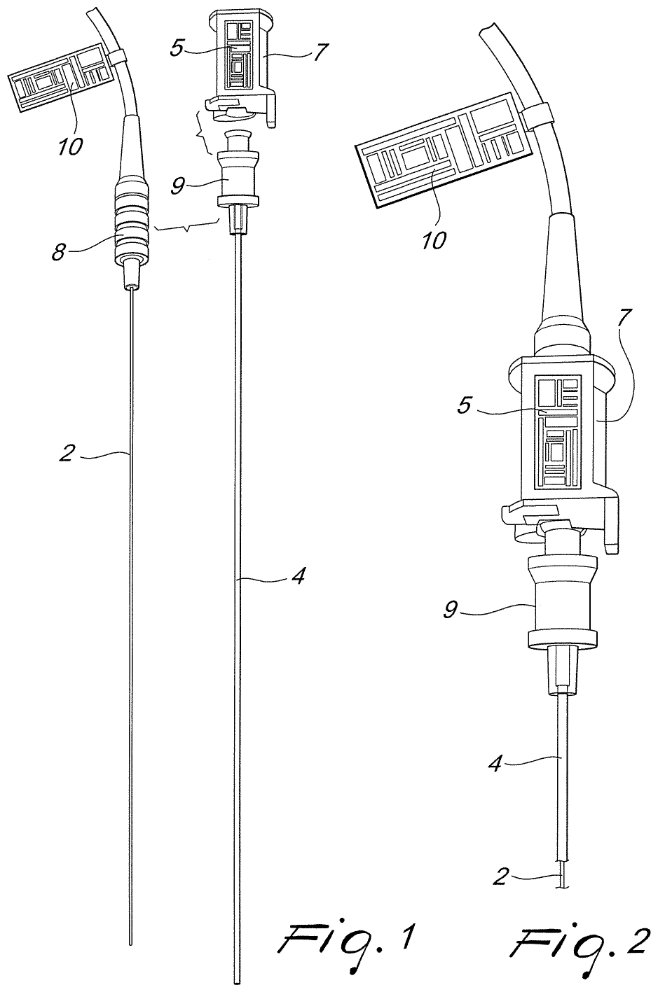

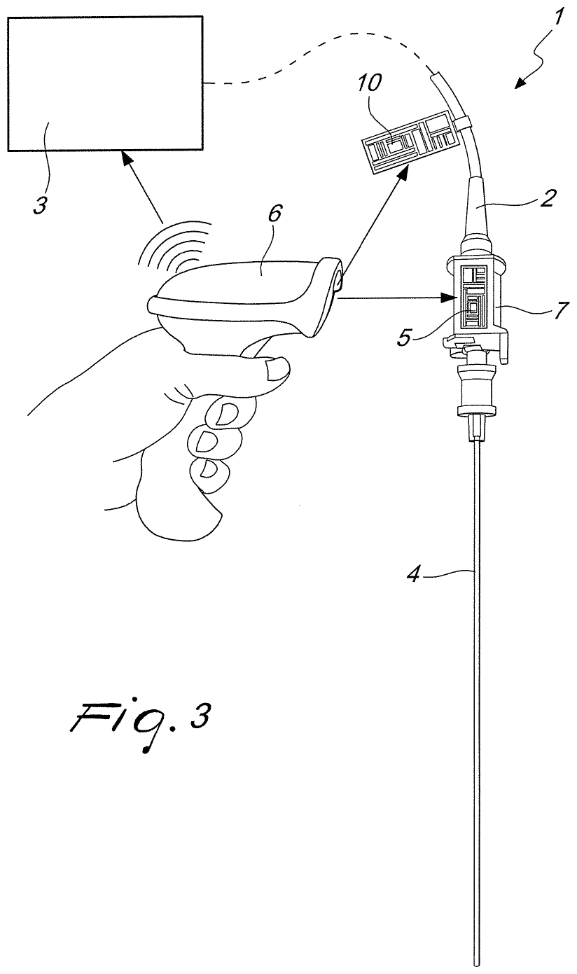

### DETAILED DESCRIPTION OF THE DRAWINGS

With reference to the figures, the reference numeral 1 generally designates a safety assembly for performing endodermal radiofrequency treatments.

Endodermal radiofrequency treatments, normally used in aesthetic is medicine to improve the appearance and firmness of the skin (although numerous other positive effects of these treatments have been tested clinically), are performed by means of a needle-shaped transducer 2 which is supplied by a respective control and management unit 3 and a cannula 4, made of electrically conducting material and partially superficially insulated, designed to accommodate the needle-shaped transducer 2 and intended to be arranged under the skin of the patient.

The transducer 2 and the cannula 4 are of a known type or in any case substantially similar to the ones already in use for endodermal radiofrequency treatments.

In practice, through a hole defined in the skin, the physician inserts the cannula 4 which contains the transducer 2 under the skin of the patient in the region to be treated: the activation of the transducer 2 entails the emission of an electrical field (the intensity and frequency of which are imposed by the control and management unit 3) which strikes the tissues of the patient that are in direct contact with at least one portion of the cannula 4.

The cannula 4 is made of electrically conducting material and its external surface is partially coated by a layer made of dielectric material: in this manner it is possible to locate the surface areas of the cannula 4 that are not coated (not electrically insulated by the layer made of dielectric material), and the radiofrequency treatment of the resistive type is delivered through these small areas.

The assembly 1 according to the disclosure comprises an identification element 5 which incorporates a first unique data string: the identification element 5 is uniquely associated with the cannula 4.

The control and management unit 3 in turn comprises a reader 6 for reading the at least one first data string from the identification element 5.

In practice, the reader 6 is a reader of a known type (which can be chosen from optical, electromagnetic, infrared, mechanical, sonic readers and a combination thereof) of a type suitable for coupling with the identification element 5.

The reader 6 is designed to enable the operation of the transducer 2 upon the reading of a correct first data string.

In particular, in relation to the fact that the cannula 4 must never be reused for subsequent treatments (for example chronologically distant treatments on a same patient or treatments on different patients), the identification element 5 associated with the cannula 4 must comprise a first data string which corresponds to a single-use activation code, which thus allows to activate the transducer 2 only upon its first use (preventing any subsequent reuses of the cannula 4, with which the identification element 5 is associated).

With particular reference to a constructive solution of unquestionable interest in practice and in application, the identification element 5 is integral with a coupling adapter 7 which comprises a through internal cavity, provided with shaped portions whose shape and dimensions are complementary to those provided on the outer surface of a grip terminal 8 of the transducer 2 and of a handle 9 of the cannula 4.

The shaped portions that are present in the internal cavity of the adapter 7 can thus be coupled to the corresponding ones of the grip terminal 8 and of the handle 9 for respective mechanical locking in a configuration of full mutual interlocking.

In this manner, the possibility that the transducer 2 might accidentally slide out of the cannula 4 during operation is avoided: this possibility would be very dangerous, since the temperature sensor of the transducer 2 (which controls in feedback the parameters of the emitted electrical field) would not be able to measure this parameter correctly and dangerous conditions for the patient might be generated.

The locking of the adapter 7 on the handle 9 is of the nondetachable type (if one tries to separate them after coupling them, at least one of the two components will be damaged): in this manner, at the end of the treatment it is possible to discard in the trash the adapter 7 and the cannula 4 mutually coupled (avoiding dangerous reuse of the cannula 4). The locking of the adapter 7 on the grip terminal 8 can instead be disassembled, in relation to the fact that the transducer 2 must be reusable for numerous treatments.

With particular reference to a constructive solution which is alternative to the one described previously, but equally functional, the identification element 5 can be advantageously integral with a grip portion of the cannula 4 (therefore, in practice, incorporated therein).

In this case, said grip portion comprises a through internal seat which leads to the internal channel of said cannula 4.

Said internal seat is positively provided with shaped portions whose shape and dimensions are complementary to those that are present on the outer surface of a grip terminal 8 of the transducer 2 for the mechanical locking of said transducer 2 in a configuration for its complete interlocking in the cannula 4. This constructive solution is substantially equivalent to the preceding one, but provides for the co-molding of the identification element 5 on the grip portion of the cannula 4 (thus minimizing the number of components of the assembly 1).

With reference to the possibility that provides for the adoption of the adapter 7, it is deemed useful to point out that the cannula 2 and the adapter 7 are coupled and are of the disposable type: this ensures, therefore, their single use once, when mutually coupled, associated with the respective transducer 2.

The sale of kits that comprise an identical number of cannulas 4 and adapters 7 forces the physician who performs the treatments to use each cannula 4 only once, together with a respective adapter 7: if one attempts to reuse a cannula 4, one is forced to pair it with a new adapter 7 (the ones that have already been used have a first data string which no longer allows the activation of the transducer 2) and therefore this is not convenient for the physician, since some cannulas 4 of the purchased kit would remain unused is (and unusable without a new adapter 7).

It is specified that the identification element 5 can be chosen preferably between a radiofrequency transceiver, a transponder, an RFID tag, and the like.

Obviously, the adoption of an identification element 5 of another type (optical, mechanical, etc.) is not excluded, although these further solutions lend themselves more to forgery with respect to a device such as an RFID tag.

It is specified furthermore that the transducer 2 can usefully comprise an identification element 10 which incorporates a second data string.

The identification element 10 also is preferably chosen from a radiofrequency transceiver, a transponder, an RFID tag, and the like.

The reader 6 controlled by the control and management unit 3 is efficiently assigned to enable the operation of the transducer 2 upon the reading of a first data string (incorporated in the identification element 5) and a second data string (incorporated in the identification element 10) which are correct and correspond.

In this manner it is possible to verify that the transducer 2 is coupled to the corresponding cannula 4 (the length of the components must be compatible, since a short transducer 2 inside a longer cannula 4 makes it impossible to control precisely the temperature with which the treatment is performed), that the cannula 4 is used only once and that the transducer 2 is used for a total treatment time that is shorter than the present maximum threshold.

The combination of these functions ensures that any danger conditions for the patient caused by reuses, incorrect couplings, excessively prolonged uses of the various components, and the like are avoided.

Furthermore, it is specified that the reader 6 controlled by the control and management unit 3 is of a type chosen from a transceiver integrated in the boxlike body of the control and management unit 3, a transceiver which can be connected to the unit 3 by means of a respective cable, a wireless transceiver apparatus which can be connected to the unit 3 remotely.

FIG. 3 shows, merely by way of example, a reader 6 wirelessly connected to the unit 3.

The possibility to use a reader 6 connected to the unit 3 by means of a respective cable with a standard terminal, for example of the USB type, is also deemed particularly interesting.

The present disclosure extends its protection also to a coupling adapter 7 for electromedical devices for the execution of endodermal radiofrequency treatments which comprise a needle-shaped transducer 2 supplied by a respective control and management unit 3 and a cannula 4, made of electrically conducting material and partially superficially insulated, designed to accommodate the needle-shaped transducer 2 during use and intended to be arranged under the skin of the patient.

The adapter 7 according to the disclosure comprises a through internal cavity provided with shaped portions the shape and dimensions of which are complementary to the ones that are present on the outer surface of the grip terminal 8 of the transducer 2 and of the handle 9 of the cannula 4 for respective locking in a configuration of full mutual interlocking.

The adapter 7 according to the disclosure comprises an identification element 5 which incorporates a unique data string. The control and management unit 3 comprises a reader 6 for reading said first data string of the identification element 5.

Said reader 6 is therefore designed to enable the operation of the transducer 2 upon a correct first data string.

The defined protective scope also extends to a method for the preventive safety checking of the components associated with a control and management unit 3 designed for the execution of endodermal radiofrequency treatments which includes a sequence of consecutive steps.

First of all it is necessary to select a transducer 2 and connect it to the is control and management unit 3.

It is then necessary to select a cannula 4 designed to accommodate the transducer 2 and couple the cannula 4 and the transducer 2 (by inserting the latter in internal in the cavity that is internal to the cannula 4).

It is then necessary to detect, by means of a respective reader 6 associated with the control and management unit 3, a first data string incorporated in an identification element 5 associated with the cannula 2, and activate the transducer 2 exclusively upon the detection of a first data string that corresponds to predefined parameters for said cannula 3 (in particular the fact that it has not been used previously for other treatments).

The method according to the disclosure may furthermore comprise a further step of detection of a second data string contained within an identification element 10 associated with the transducer 2.

The further step of detection of the second data string is also necessary for the activation of the transducer 2, which is dependent upon the detection of said second data string which corresponds to predefined parameters for the transducer 2 (for example a total operating time below a preset threshold) and to the correct pairing of the cannula 4 with the transducer 2 (components having corresponding dimensions).

It should be specified that with reference to the cited method, the identification element 5 associated with the cannula 4 can favorably be coupled to an adapter 7 which can be attached stably to the cannula 4. Said adapter 7 is thus provided with means for detachable coupling to the transducer 2 and can instead be associated uniquely with a respective cannula 4.

The use of an adapter 7 is particularly advantageous since it allows the physician (or in general the operator) to grip it stably for the movement of the assembly constituted by the adapter 7, by the transducer 2 and by the cannula 4, in order to perform all the operations required for the treatment. Better grip allows the physician (or the operator) to place the cannula 4 with greater precision, optimizing the treatment.

Advantageously, the present disclosure solves the problems described previously, proposing a safety assembly 1 for the execution of endodermal radiofrequency treatments that ensures the single use of each needle cannula 2, safeguarding the safety of the patients.

The presence of the identification element 5 on which the authorization for operation depends ensures the elimination of the mentioned risk.

Usefully, the safety assembly 1 according to the disclosure ensures the correct replacement of each transducer 2 when it has reached the respective expected life limit.

The presence of the identification element 10 in fact allows to monitor the overall duration of the treatments performed by the transducer 2 and to interrupt its operation if the reader 6 detects the passing of a preset threshold value.

Conveniently, the safety assembly 1 according to the disclosure ensures the optimum and correct coupling of each individual transducer 2 to the corresponding needle cannula 4.

The shaped portions that are internal to the adapter 7 in fact produce stable couplings to the grip terminal 8 of the transducer 2 and to the handle 9 of the cannula 4.

Validly, the safety assembly 1 according to the disclosure is relatively simple to provide in practice and has substantially a low cost: these characteristics make the assembly 1 according to the disclosure an innovation of assured application.

The disclosure thus conceived is susceptible of numerous modifications and variations, all of which are within the scope of the appended claims; all the details may furthermore be replaced with other technically equivalent elements.

In the exemplary embodiments shown, individual characteristics, given in relation to specific examples, may actually be interchanged with other different characteristics that exist in other exemplary embodiments.

In practice, the materials used, as well as the dimensions, may be any according to the requirements and the state of the art.

1-11. (canceled)

12. A safety assembly for performing endodermal radiofrequency treatments, performed by a needle-shaped transducer supplied by a respective control and management unit and a cannula, made of electrically conducting material and partially superficially insulated, designed to accommodate said needle-shaped transducer and configured to be arranged under the skin of a patient, comprising an identification element, uniquely associated with said cannula, which incorporates a first unique data string, said control and management unit comprising a reader for reading at least said first data string of said identification element, said reader being designed to enable the operation of said transducer upon reading a correct first data string.

13. The safety assembly according to claim 12, wherein said identification element is integral with a coupling adapter which comprises a through internal cavity provided with shaped portions whose shape and dimensions are complementary to those that are present on an outer surface of a grip terminal of said transducer and of a handle of said cannula, for respective mechanical locking in a configuration of full mutual interlocking.

14. The safety assembly according to claim 12, wherein said identification element is integral with a grip portion of said cannula, said grip portion comprising a through internal seat which leads to the internal channel of said cannula, said internal seat being provided with shaped portions whose shape and dimensions are complementary to those that are present on an outer surface of a grip terminal of said transducer for a mechanical locking of said transducer in a configuration of full interlocking in the cannula.

15. The safety assembly according to claim 13, wherein said cannula and said adapter are coupled and are disposable, for their single use once they have been mutually coupled and associated with the respective transducer.

16. The safety assembly according to claim 12, wherein said identification element is chosen between at least a radiofrequency transceiver, a transponder, and an RFID tag.

17. The safety assembly according to claim 12, wherein said transducer comprises an identification element which incorporates a second data string, said identification element being chosen from at least a radiofrequency transceiver, a transponder, and an RFID tag, said reader being designed to enable operation of said transducer upon a reading of correct and corresponding first and second data strings.

18. The safety assembly according to claim 12, wherein said reader is chosen from a transceiver integrated in a box-like body of the control and management unit, a transceiver apparatus which can be connected to said unit by means of a respective cable, and a wireless transceiver apparatus which can be connected to said unit remotely.

19. A coupling adapter for electromedical devices for executing endodermal radiofrequency treatments, comprising a needle-shaped transducer supplied by a respective control and management unit and a cannula, made of an electrically conducting material and partially superficially insulated, designed for the accommodation of said needle-shaped transducer and configured to be arranged under the skin of a patient, further comprising a through internal cavity which is provided with shaped portions whose shape and dimensions are complementary to those that are present on an outer surface of a grip terminal of said transducer and of a handle of said cannula for respective locking in a configuration for full mutual interlocking, said adapter comprising an identification element which incorporates a unique data string, said control and management unit comprising a reader of at least said data string of said identification element, said reader being designed to enable the operation of said transducer upon a correct data string.

20. A method for the preventive safety checking of components associated with a control and management unit designed to perform endodermal radiofrequency treatments, the method including the following steps:
selecting a transducer and connecting it to said control and management unit,
selecting a cannula designed to accommodate said transducer and to couple the cannula and the transducer, and
detecting, by means of a respective reader associated with said control and management unit, a first data string which is incorporated in an identification element associated with said cannula and activating said transducer only following the detection of a first data string that corresponds to predefined parameters for said cannula.

21. The method according to claim 20, wherein said detection step includes detecting a second data string contained within an identification element associated with said transducer, the activation of said transducer being dependent upon the detection also of a second data string that corresponds to predefined parameters for said transducer and to the correct pairing of said cannula with said transducer.

22. The method according to claim 20, wherein said identification element associated with said cannula is coupled to an adapter which can be coupled stably to said cannula and is provided with means for detachable coupling to said transducer, said adapter being uniquely associable with a respective cannula.
